(19) United States
(12) Patent Application Publication
(10) Pub. No.: US 2009/0082801 A1
(43) Pub. Date: Mar. 26, 2009

(54) Tool for inserting a receiver of a hearing apparatus into an auditory canal

(75) Inventors: Ulrich Giese, Erlangen (DE); Sebastian Pape, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL INSTRUMENTS PTE. LTD.

(21) Appl. No.: 12/284,141

(22) Filed: Sep. 18, 2008

(30) Foreign Application Priority Data
Sep. 21, 2007 (DE) 10 2007 045 315.0

(51) Int. Cl. A61B 17/50

(52) U.S. Cl. 606/210

(57) ABSTRACT

The insertion of an external receiver of a hearing apparatus and in particular a hearing device into an auditory canal of a user is to be facilitated. Provision is made for this purpose for a tool comprising a pair of tweezers with a first and a second tweezer arm, which are affixed to one another at one end. The first tweezer arm has a holding device at its free end by means of which the movements of the receiver to be held are restricted in at least three directions. Furthermore at least one of the two tweezer arms has a stopper that stands substantially perpendicular to the tweezer arm and serves to abut against a concha of the user during insertion of the receiver. In this way the external receiver can not only be grasped well but also inserted into the auditory canal to a defined depth.

### CROSS REFERENCE TO RELATED APPLICATIONS

This application claims priority of German application No. 10 2007 045 315.0 filed Sep. 21, 2007, which is incorporated by reference herein in its entirety.

### FIELD OF THE INVENTION

The present invention relates to a tool for inserting a receiver of a hearing apparatus into an auditory canal, comprising a pair of tweezers with a first and a second tweezer arm, which are affixed to one another at one end. The term “hearing apparatus” is understood here to mean in particular a hearing device, but also any other device that can be worn on/in the ear for emitting sound such as a headset, a set of ear phones and the like.

### BACKGROUND OF THE INVENTION

Hearing devices are wearable hearing apparatuses which are used to assist the hard-of-hearing. In order to accommodate numerous individual requirements, various types of hearing devices are available such as behind-the-ear (BTE) hearing devices, hearing device with external receiver (RIC: receiver in the canal) and in-the-ear (ITE) hearing devices, for example also concha hearing devices or completely-in-the-canal (ITE, CIC) hearing devices. The hearing devices listed as examples are worn on the outer ear or in the auditory canal. Bone conduction hearing aids, implantable or vibrotactile hearing aids are also available on the market. The damaged hearing is thus stimulated either mechanically or electrically.

The key components of hearing devices are principally an input converter, an amplifier and an output converter. The input converter is normally a receiving transducer e.g. a microphone and/or an electromagnetic receiver, e.g. an induction coil. The output converter is most frequently realized as an electroacoustic converter e.g. a miniature loudspeaker, or as an electromechanical converter e.g. a bone conduction hearing aid. The amplifier is usually integrated into a signal processing unit. This basic configuration is illustrated in FIG. 1 using the example of a behind-the-ear hearing device. One or a plurality of microphones 2 for recording ambient sound are built into a hearing device housing 1 to be worn behind the ear. A signal processing unit 3 which is also integrated into the hearing device housing 1 processes and amplifies the microphone signals. The output signal for the signal processing unit 3 is transmitted to a loudspeaker or receiver 4, which outputs an acoustic signal. Sound is transmitted through a sound tube, which is affixed in the auditory canal by means of an otoplastic, to the device wearer's eardrum. Power for the hearing device and in particular for the signal processing unit 3 is supplied by means of a battery 5 which is also integrated in the hearing device housing 1.

In order to wear a RIC hearing device it is necessary to insert into the auditory canal the receiver that is separate from the hearing device housing, and to affix to the concha the hearing device housing that is connected to the receiver by means of a cable. Experience has shown that hearing device wearers find it difficult, at least at the beginning, to insert the receiver of the RIC hearing device correctly into the auditory canal. In many cases this compromises the level of spontaneous acceptance of RIC hearing devices. The receiver can only be inserted into the auditory canal with difficulty because the receiver is itself very small and can only be pushed sufficiently far into the auditory canal with a very high level of fingertip control. Consequently the receiver is generally pushed back and forth with the aid of the connected cable until it is felt that the receiver is positioned correctly. However the hearing device wearer continues to feel uncertain as to whether or not the receiver is positioned sufficiently deeply in the ear.

### SUMMARY OF THE INVENTION

The object of the present invention is thus to facilitate the insertion of a receiver into an auditory canal.

This object is inventively achieved by means of a tool for inserting a receiver of a hearing apparatus into an auditory canal of a wearer, said tool comprising a pair of tweezers with a first and a second tweezer arm, which are affixed to one another at one end, with the first tweezer arm having a holding device at its free end by means of which the movements of the receiver to be held are restricted in at least three directions, and with one of the two tweezer arms having a stopper that stands substantially perpendicular to the tweezer arm and serves to abut against a concha of the user during insertion of the receiver.

The user and/or hearing device wearer can advantageously by means of the inventive tool not only grasp securely but also insert the receiver into the auditory canal to a defined depth.

The aforementioned holding device preferably has a U-shaped cross-section. The receiver can easily be engaged in a holding recess shaped in this way and can be squeezed tight by means of the second tweezer arm.

Furthermore it can be advantageous if an aforementioned stopper is arranged on each of the tweezer arms. This can further facilitate the insertion of the receiver and prevent the receiver being pushed too far into the auditory canal.

Furthermore the pair of tweezers can have a bend along its length that is transverse to the direction of movement of the tweezer arms. This shape of tweezers enables ergonomic considerations to be accommodated better.

According to a further embodiment the tool can be made of a plastic. In this way not only the weight of the tool but also the risk of damage to the receiver can be reduced.

Furthermore the free end of one or both of the tweezer arms can have a rubber coating. As a result the receiver can be grasped and held better.

### BRIEF DESCRIPTION OF THE DRAWINGS

The present invention is described in more detail with reference to the appended drawings, in which

FIG. 1 shows a basic sketch of the configuration of a hearing device according to the prior art;

FIG. 2 shows a basic sketch of a tool according to the invention viewed from the side;

FIG. 3 shows a perspective view of a receiver to be inserted into an auditory canal;

FIG. 4 shows the tool shown in FIG. 2 from the front; and

FIG. 5 shows a plan view of an inventive tool according to a special embodiment.

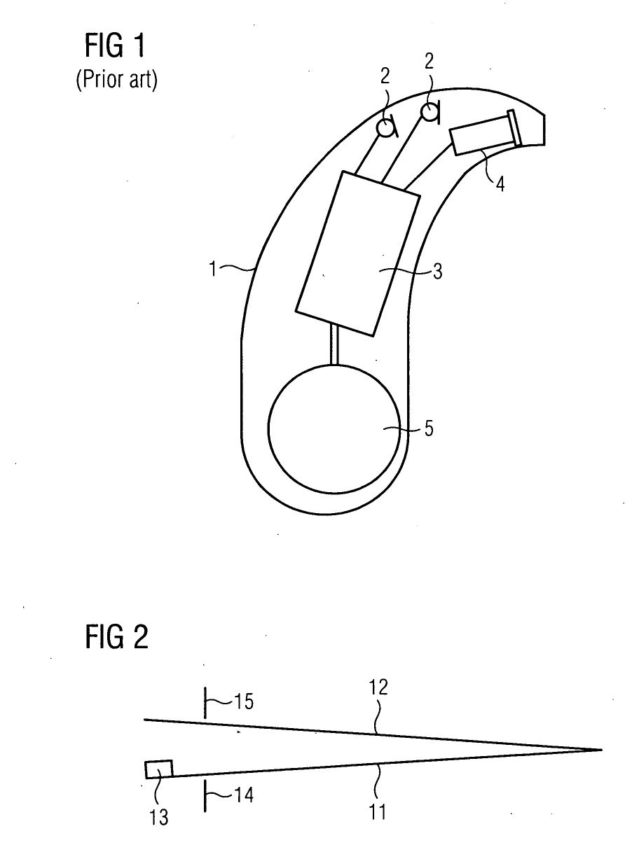

### DETAILED DESCRIPTION OF THE INVENTION

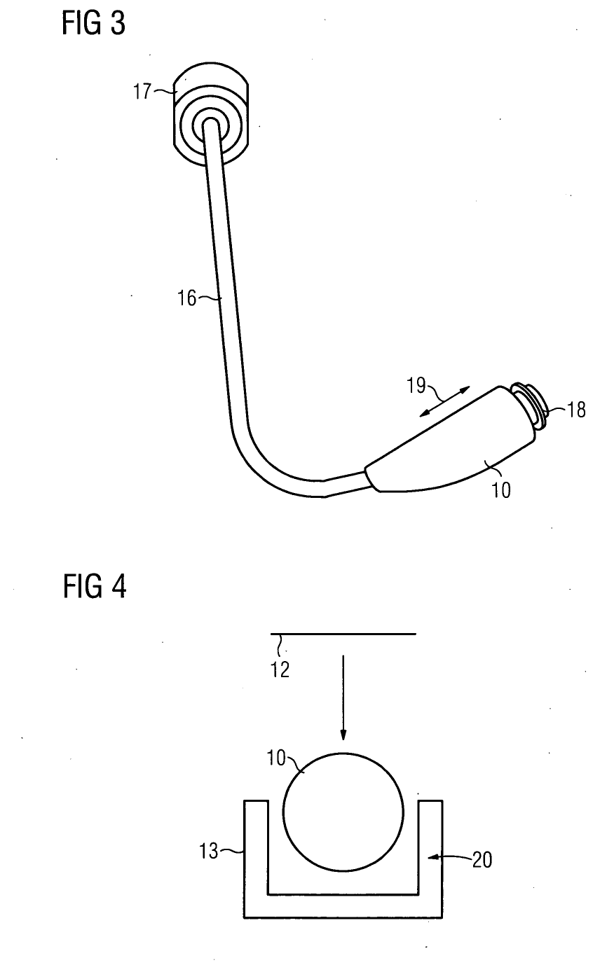

The basic idea of the present invention consists in providing a tool that assists the hearing device wearer to insert the external receiver 10 (cf. FIG. 3) into the auditory canal. With such a tool it is then possible to hold the receiver more securely and introduce it into the ear in a more controlled fashion.

In accordance with the exemplary embodiment of FIG. 2 the tool is essentially constructed in accordance with the principle of a pair of tweezers. The tool has a first tweezer arm 11 and a second tweezer arm 12. The tweezer arms 11, 12 are each connected to one another at one end. They are furthermore movable towards one another in a spring-elastic fashion.

The first tweezer arm 11 has at its free end a recess 13 with a U-profile as a holding device for the receiver 10. Each of the two tweezer arms 11, 12 further has a stopper 14, 15 that extends substantially perpendicular to the corresponding tweezer arm 11, 12. In principle a single stopper on one of the two tweezer arms is also sufficient. This stopper or these stoppers prevent the receiver from being capable of being inserted too deeply into the auditory canal with the tool. The stoppers 14, 15 can accordingly also be designed to be adjustable so that the acoustician for example can configure the correct depth of insertion for the hearing device wearer. The configuration can be performed e.g. by means of a retaining screw or a thread on the upper surface of one of the tweezer arms.

A typical external receiver 10 is shown in FIG. 3. Said receiver is connected with the aid of a cable 16 to a hearing device housing 17 (partially shown). At the output of the receiver is located a mount 18 (so-called “receiver spout”) onto which an earmold piece (so-called “dome”—not shown) can be mounted. This earmold piece generally also contains a cerumen protection system. The dome can slip off the mount at the output of the receiver or the cerumen protection system can disengage from the dome precisely in the event of incorrect handling and/or insertion and removal of the hearing device into/from the auditory canal. In order to avoid this eventuality and also in order to grasp the receiver securely and insert it into the auditory canal to a predetermined depth, the hearing device wearer can now engage the tool shown in FIG. 2. To this end he should grasp the receiver 10 with the recess 13 in the region 19, in other words in its mid-region.

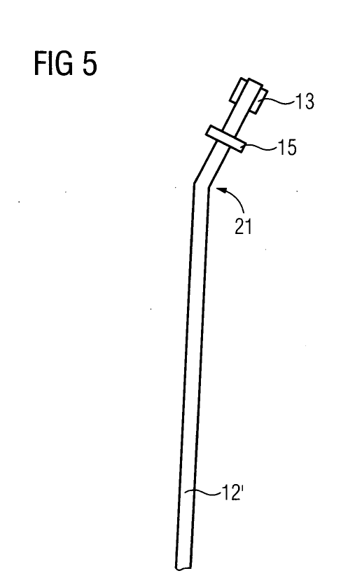

FIG. 4 shows a front view of the point of the tool shown in FIG. 2. The receiver 10 is held in the recess 13, which has a U-shaped profile. Accordingly the receiver 10 is secured from three sides: from below, from the left and from the right. The receiver is squeezed into the recess 13 with the aid of the second tweezer arm 12. In this way its movement is also restricted in the remaining three directions of movement: upwards, as well as in both axial directions of the receiver 10 (perpendicular to the level of the drawing). In both of these axial directions the receiver 10 is held by friction. In order to increase this friction the recess 13 is preferably provided with a layer of rubber 20. A rubber coating of this kind can also be provided on the second tweezer arm 12.

FIG. 5 shows a plan view of a tweezer-shaped tool according to a special embodiment. The pair of tweezers has a bend 21 along its longitudinal axis. The plan view in FIG. 5 shows the bent upper tweezer arm 12′ including the stopper 15. Only the recess 13 of the lower tweezer arm, which is also bent, can be seen. At the point of the tweezers, roughly one third of their length is bent to the side by approximately 20°-30°, in other words in a perpendicular direction to the direction of movement of the tweezer arms. The inclination can be to the left or to the right in order to facilitate insertion of the receiver into the left ear and/or into the right ear. Of course the angle of inclination can also be any other angle preferably between 0° and 90°. In any case the ergonomic considerations of the tool will be accommodated by the bend 21 and the inclination.

The tool is preferably made of a rigid plastic or metal.

1-6. (canceled)

7. A tool for inserting a receiver of a hearing apparatus into an auditory canal of a user, comprising:
a pair of tweezers comprising a first tweezer arm and a second tweezer arm that are affixed to one another at one end of the first tweezer arm and the second tweezer arm;
a holding device arranged at a free end of the first tweezer arm that restricts a movement of the receiver; and
a stopper arranged perpendicularly at one of the first tweezer arm and the second tweezer arm that abuts against a concha of the user during the insertion of the receiver.

8. The tool as claimed in claim 7, wherein the holding device comprises a U-shaped cross-section.

9. The tool as claimed in claim 7, wherein the stopper is arranged at each of the first tweezer arm and the second tweezer arm.

10. The tool as claimed in claim 7, wherein the pair of tweezers comprises a bend along a longitudinal axis that is transverse to a direction of a movement of the first tweezer arm and the second tweezer arm.

11. The tool as claimed in claim 7, wherein the tool is made of a plastic.

12. The tool as claimed in claim 7, wherein the free end of the first tweezer arm comprises a rubber coating.

13. The tool as claimed in claim 7, wherein a free end of the second tweezer arm comprises a rubber coating.
